(19) United States
(12) Patent Application Publication
(10) Pub. No.: US 2011/0245536 A1
(43) Pub. Date: Oct. 6, 2011

(54) PROCESS FOR PREPARING PREGABALIN

(75) Inventors: Vikas Daulatrao Ahirrao, Maharashtra (IN); Depashri Vikas Ahirrao, Maharashtra (IN); Chandra Prakash Narani, Andhra Pradesh (IN); Sandipan Prabhurao Bondge, Maharashtra (IN); Mayur Devjibhai Khunt, Gujarat (IN); Nitin Sharadchandra Pradhan, Maharashtra (IN); Jon Valgeirsson, Hafnarfjordur (IS)

(73) Assignee: ACTAVIS GROUP PTC EHF, Hafnarfjordur (IS)

(21) Appl. No.: 12/995,782

(22) PCT Filed: Jun. 1, 2009
(86) PCT No.: PCT/IB2009/005995

§ 371 Date: May 23, 2011

(30) Foreign Application Priority Data
Jun. 2, 2008 (IN) 1342/CHE/2008

(51) Int. Cl. C07C 227/40; C07C 227/42

(52) U.S. Cl. 562/554

(57) ABSTRACT

Disclosed herein is an improved, commercially viable and industrially advantageous process for the preparation of pregabalin in high yield and purity. The present invention also provides a process for the purification of (S)-pregabalin.

### CROSS REFERENCE TO RELATED APPLICATION

This application claims the benefit of priority to Indian provisional application No. 1342/CHE/2008, filed on Jun. 2, 2008, which is incorporated herein by reference in its entirety.

### FIELD OF THE DISCLOSURE

Disclosed herein is an improved, commercially viable and industrially advantageous process for the preparation of pregabalin in high yield and purity. Also provided is a process for the purification of (S)-pregabalin.

### BACKGROUND

Pregabalin or racemic pregabalin, chemically named 3-(aminomethyl)-5-methylhexanoic acid, is indicated for the management of neuropathic pain associated with diabetic peripheral neuropathy, management of posterpetic neuralgia, adjunctive therapy for adult patients with partial onset seizures, and management of fibromyalgia. Pregabalin is an analog of 4-aminobutyric acid (GABA), a neurotransmitter that is thought to play a major inhibitory role in the central nervous system (CNS). Pregabalin is represented by the following structural formula:

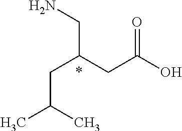

The structural formula of pregabalin contains one chiral centre (the asterisk designates the chiral centre) and thus exists as two optical isomers, i.e., enantiomers (R- & S-isomers). (S)-Pregabalin, chemically named (S)-(+)-3-(aminomethyl)-5-methylhexanoic acid, is represented by the following structural formula:

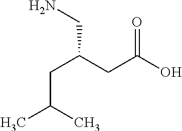

(S)-Pregabalin is available under the trade name LYRICA® in tablets for 25, 50, 75, 100, 150, 200, 225, 300 mg doses. (S)-Pregabalin has been found to activate GAD (L-glutamic acid decarboxylase), has a dose dependent protective effect on seizures, and is a CNS-active compound. (S)-Pregabalin has been found to be useful in anticonvulsant therapy, due to its activation of GAD, promoting the production of GABA, one of the brain's major inhibitory neurotransmitters, which is released at 30% of brain synapses.

Various processes for the preparation of pregabalin and related compounds are disclosed in U.S. Pat. Nos. 5,599,973 and 5,616,793, and PCT Publication No. WO 2006/122258.

U.S. Pat. No. 5,599,973 (hereinafter referred to as the '973 patent) describes two synthetic routes for preparing pregabalin. These routes each involve reactions that require n-butyllithium, and each route contains a step that must be carried out at low temperatures (≦−35° C.) under carefully controlled conditions. Theses synthetic routes include the use of (4R,5S)-4-methyl-5-phenyl-2-oxazolidinone as a chiral auxiliary to introduce the stereochemical configuration needed in the final product.

The synthetic routes described in the '973 patent suffers from disadvantages such as high cost of reagents, the use of pyrophoric and explosive reagents like n-butyllithium, the use of additional reagents, low yields of product, low temperatures, and health hazards. Hence, these routes are not advisable for scale up operations.

U.S. Pat. No. 5,616,793 (hereinafter referred to as the '793 patent) discloses a process for preparing (S)-pregabalin via a Hoffmann degradation of (R)-(−)-3-(carbamoylmethyl)-5-methylhexanoic acid with Br₂/NaOH, followed by precipitation of (S)-pregabalin, after addition of hydrochloric acid. The '793 patent further describes a process for the purification of (S)-pregabalin by crystallization from a mixture of isopropanol and water.

PCT Publication No. WO 2006/122258 (hereinafter referred to as the '258 application) describes a process for the preparation of pregabalin comprising combining an alkali hydroxide and water; adding 3-(carbamoylmethyl)-5-methylhexanoic acid at a temperature of about 0° C. to about 40° C.; adding bromine, in a drop-wise manner, at a temperature of about 0° C. to about 40° C.; heating the reaction mixture; reacting with a strong mineral acid; extracting with a C4-8 alcohol, and mixing with a base.

The processes described in both the '793 patent and '258 application involve the use of strong mineral acids such as hydrochloric acid and sulfuric acid. The use of strong mineral acids in the processes corrodes the equipment used in manufacture. Moreover, the handling of these chemicals is also difficult in the plant scale and hence, the processes are not advisable for scale up operations. In addition, the purification process described in the '793 patent does not produce a product with satisfactory purity since the process does not reduce or eliminate the impurities formed during the manufacturing process.

Based on the aforementioned drawbacks, the prior art processes may be unsuitable for preparation of pregabalin or the enantiomers thereof in commercial scale operations.

A need remains for an improved and commercially viable process of preparing a substantially pure pregabalin and its enantiomers, or a pharmaceutically acceptable salt thereof, to resolve the problems associated with the processes described in the prior art, and that will be suitable for large-scale preparation, in a shorter reaction time. Desirable process properties include less hazardous and environmentally friendly reagents, reduced cost, greater simplicity, increased product purity and increased yield of the product.

### SUMMARY

The present inventors have surprisingly found that pregabalin or an (S)-enantiomer thereof can be prepared in high purity and with high yield by admixing 3-(carbamoylmethyl)-5-methylhexanoic acid or an (R)-enantiomer thereof with an alkali hydroxide solution in water at a temperature of about −5° C. to about −15° C.; adding bromine, in a drop-wise manner, at a temperature of about −5° C. to about −15° C.; heating the resulting mass to a temperature of about 40° C. to about 90° C.; optionally cooling to a temperature of below 40° C.; reacting the resulting mass with an organic acid; extracting with an alcoholic solvent, and treating with a base.

In another aspect, provided also herein is a process for the purification of pregabalin or an (S)-enantiomer thereof by recrystallizing the product from a solvent medium comprising methanol and water, wherein the ratio of methanol to water is 6-8:2-4.

Described herein is an efficient, convenient, commercially viable and environment friendly process for the preparation of pregabalin and its enantiomers, or a pharmaceutically acceptable salt thereof. The process described herein avoids the use of strong mineral acids, thereby avoiding the tedious and cumbersome procedures of the prior art, and is convenient to operate on a commercial scale.

Advantageously, the reagents used for the method described herein are less hazardous and easier to handle at a commercial scale, and are also less expensive reagents than those used previously.

### DETAILED DESCRIPTION

The term “pregabalin” in all aspects of the present disclosure, refers to either the S-enantiomer or the racemate of 3-(aminomethyl)-5-methyl-hexanoic acid.

As used herein, unless specified otherwise, when racemic 3-(carbamoylmethyl)-5-methylhexanoic acid is used as a starting material, the product obtained is pregabalin racemate; and when (R)-3-(carbamoylmethyl)-5-methylhexanoic acid is used as a starting material, the product obtained is (S)-pregabalin.

According to one aspect, there is provided an improved process for the preparation of pregabalin or an (S)-enantiomer thereof, comprising:
a) admixing 3-(carbamoylmethyl)-5-methylhexanoic acid or an (R)-enantiomer thereof with a solution of alkali metal hydroxide in water at a temperature of about −5° C. to about −15° C. to form an admixture;
b) adding bromine to the admixture obtained step-(a) at a temperature of about −5° C. to about −15° C. to form a reaction mixture;
c) heating the reaction mixture obtained in step-(b) at a temperature of about 40° C. to about 100° C. to form a first reaction mass;
d) reacting the first reaction mass obtained step-(c) with an organic acid to form an organic acid addition salt of pregabalin;
e) optionally, extracting the organic acid addition salt of pregabalin obtained in step-(d) with an organic solvent to provide an organic layer;
f) treating the product obtained in step-(d) or the organic layer obtained in step-(e) with a base to form a second reaction mass;
g) heating the second reaction mass obtained in step-(f) at a temperature of about 40° C. to about 100° C. to produce a third reaction mass; and
h) isolating substantially pure pregabalin or an (S)-enantiomer thereof from the third reaction mass, and optionally recrystallizing the substantially pure pregabalin or an (S)-enantiomer thereof obtained from a solvent to produce highly pure pregabalin or an (S)-enantiomer thereof.

The total purity of the pregabalin or its (S)-enantiomer obtained by the process disclosed herein is greater than about 99%, specifically greater than about 99.5%, and more specifically greater than about 99.9% as measured by HPLC. In one embodiment, the purity of the pregabalin or its (S)-enantiomer is about 99% to about 99.95%, or about 99.5% to about 99.99%.

In one embodiment, the alkali metal hydroxide used in step-(a) is selected from the group consisting of sodium hydroxide, potassium hydroxide, lithium hydroxide and cesium hydroxide. A specific alkali metal hydroxide is sodium hydroxide.

The admixing in step-(a) is done in a suitable order, for example, the 3-(carbamoylmethyl)-5-methylhexanoic acid or its (R)-enantiomer is added to the alkali metal hydroxide solution, or alternatively, the alkali metal hydroxide solution is added to the 3-(carbamoylmethyl)-5-methylhexanoic acid or its (R)-enantiomer. The addition is, for example, carried out drop wise, in one portion, or in more than one portion. The addition is specifically carried out at a temperature of about −5° C. to about −12° C. for at least 10 minutes, and more specifically at about −5° C. to about −10° C. for about 20 minutes to about 2 hours under stirring.

In one embodiment, the alkali metal hydroxide used in step-(a) is in a molar ratio of about 4 to 8 moles, specifically about 4.5 to 5.5 moles, per mole of 3-(carbamoylmethyl)-5-methylhexanoic acid or its (R)-enantiomer.

In another embodiment, the addition of bromine in step-(b) is carried out in a drop-wise manner. The addition is specifically carried out at a temperature of about −5° C. to about −12° C. for at least 20 minutes and more specifically at a temperature of about −5° C. to about −10° C. for about 30 minutes to about 4 hours.

In one embodiment, the bromine used in step-(b) is in a molar ratio of about 1 to 3 moles, specifically about 1.05 to 1.25 moles, per mole of 3-(carbamoylmethyl)-5-methylhexanoic acid or its (R)-enantiomer.

The reaction mixture in step-(c) is specifically heated at a temperature of about 50° C. to about 90° C. for at least 15 minutes and more specifically at about 60° C. to about 85° C. for about 20 minutes to about 2 hours to form the first reaction mass. The first reaction mass obtained after completion of reaction in step-(c) is specifically cooled at a temperature of below 40° C., and more specifically at a temperature of about 10° C. to about 30° C.

Exemplary organic acids used in step-(d) include, but are not limited to, oxalic acid, methanesulfonic acid, trifluoroacetic acid, benzenesulfonic acid, and the like. Specific organic acids are oxalic acid and methanesulfonic acid.

The reaction in step-(d) is specifically carried out at a temperature of below 40° C. for at least 15 minutes, and more specifically at a temperature of about 10° C. to about 30° C. for about 20 minutes to about 2 hours. In one embodiment, a pH of less than about 3, specifically less than about 2, and most specifically less than about 1, is obtained when the organic acid is added.

In one embodiment, the product containing the organic acid addition salt of pregabalin obtained in step-(d) is subjected to usual work up such as washings, extractions, evaporations etc. In another embodiment, the product may be used directly in the next step to produce the pregabalin or the organic acid addition salt of pregabalin may be isolated and then used in the next step.

Specifically, the organic acid addition salt of pregabalin obtained in step-(d) is purified without isolating it. In one embodiment, this salt is purified by selective extractions with an organic solvent selected from the group consisting of alcohols, ketones, esters and the like and mixtures thereof.

In one embodiment, the extracting solvent used in step-(e) is selected from the group consisting of methanol, ethanol, n-propanol, isopropyl alcohol, isobutanol, n-butanol, tert-butanol, amyl alcohol, isoamyl alcohol, hexanol, acetone, methyl ethyl ketone, methyl isobutyl ketone, methyl tert-butyl ketone, ethyl acetate, methyl acetate, isopropyl acetate, tert-butyl methyl acetate, ethyl formate, and mixtures thereof.

Specifically, the extracting solvent is selected from the group consisting of isobutanol, n-butanol, tert-butanol, amyl alcohol, isoamyl alcohol, hexanol, and mixtures thereof; and more specifically isobutanol.

The organic layer obtained in step-(e) is optionally cooled at a temperature of about 0° C. to about 10° C., followed by filtering off the inorganic salts obtained in the reaction.

The base used in step-(f) is an organic or inorganic base. Exemplary organic bases are triethyl amine, tributyl amine, ammonia, diisopropyl amine, dimethyl amine and diisopropyl ethyl amine; and more specifically triethyl amine, tributyl amine, ammonia and diisopropyl ethyl amine.

Exemplary inorganic bases include, but are not limited to, hydroxides and carbonates of alkali metals. Specific inorganic bases are sodium hydroxide, potassium hydroxide, lithium hydroxide, sodium carbonate, potassium carbonate, lithium carbonate; and more specifically sodium hydroxide, potassium hydroxide, sodium carbonate and potassium carbonate.

The reaction mass in step-(g) is specifically heated at a temperature of about 50° C. to about 90° C. for at least 15 minutes, and more specifically at about 60° C. to about 85° C. for about 20 minutes to about 2 hours.

The isolation in step-(h) is initiated by a method usually known in the art such as cooling, seeding, partial removal of the solvent from the solution, by adding an anti-solvent to the solution, or a combination thereof.

In one embodiment, the isolation is carried out by cooling the solution under stirring at a temperature of below 25° C. for at least 10 minutes, specifically at about 0° C. to about 25° C. for about 20 minutes to about 20 hours, and more specifically at about 0° C. to about 10° C. for about 30 minutes to about 10 hours.

The pregabalin or its (S)-enantiomer obtained in step-(h) is recovered by methods such as filtration, filtration under vacuum, decantation, centrifugation, or a combination thereof. In one embodiment, pregabalin or its (S)-enantiomer is recovered by filtration employing a filtration media of, for example, a silica gel or celite.

In one embodiment, the recrystallization in step-(h) is carried out by methods disclosed hereinafter.

In another embodiment, the recrystallization solvent used in step-(h) is an aqueous alcohol solvent. A specific recrystallization solvent is an aqueous methanol solvent, characterized in that wherein the ratio of methanol to water is of 6-8:2-4.

The pure pregabalin or its (S)-enantiomer obtained by the process disclosed herein may be further dried in, for example, a Vacuum Tray Dryer, Rotocon Vacuum Dryer, a Vacuum Paddle Dryer or a pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In one embodiment, drying is carried out at atmospheric pressure or reduced pressures, such as below about 200 mm Hg, or below about 50 mm Hg, at temperatures such as about 35° C. to about 90° C. The drying can be carried out for any desired time period that achieves the desired result, such as about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer and the like. Drying equipment selection is well within the ordinary skill in the art.

According to another aspect, there is provided a process for purifying (S)-pregabalin, comprising:
a) providing a solution of crude (S)-pregabalin in a solvent medium comprising methanol and water, wherein the ratio of methanol to water is 6-8:2-4;
b) optionally, filtering the solution; and
c) isolating a highly pure (S)-pregabalin from the solution.

In one embodiment, step-(a) of providing a solution of crude (S)-pregabalin includes dissolving crude (S)-pregabalin in the solvent, or obtaining an existing solution from a previous processing step.

In one embodiment, the crude (S)-pregabalin is dissolved in the solvent medium at a temperature of about 40° C. to the reflux temperature of the solvent medium used, specifically at about 50° C. to about 80° C., and more specifically at about 55° C. to about 75° C.

In another embodiment, the solution obtained in step-(a) is stirred at a temperature of about 50° C. to about 80° C. for at least 20 minutes, and specifically at a temperature of about 55° C. to about 75° C. for about 30 minutes to about 5 hours.

The solution obtained in step-(a) is optionally subjected to carbon treatment or silica gel treatment. The carbon treatment or silica gel treatment is carried out by methods known in the art, for example, by stirring the solution with finely powdered carbon or silica gel at a temperature of below about 80° C. for at least 15 minutes, specifically at a temperature of about 40° C. to about 70° C. for at least 30 minutes; and filtering the resulting mixture through hyflo to obtain a filtrate containing (S)-pregabalin by removing charcoal or silica gel. Specifically, the finely powdered carbon is an active carbon. A specific mesh size of silica gel is 40-500 mesh, and more specifically 60-120 mesh.

The isolation of pure (S)-pregabalin in step-(c) is initiated by a method usually known in the art such as cooling, seeding, partial removal of the solvent from the solution, by adding an anti-solvent to the solution, or a combination thereof.

In one embodiment, the isolation is carried out by cooling the solution under stirring at a temperature of below 25° C., specifically at about 0° C. to about 20° C. for about 20 minutes to about 20 hours, and more specifically at about 0° C. to about 10° C. for about 30 minutes to about 10 hours.

The highly pure (S)-pregabalin obtained in step-(c) is recovered and further dried by the methods described hereinabove.

The total purity of the (S)-pregabalin obtained by the process disclosed herein is greater than about 99%, specifically greater than about 99.90%, and more specifically greater than about 99.95% as measured by HPLC. For example, the purity of the (S)-pregabalin can be about 99% to about 99.95%, or about 99.5% to about 99.99%. The term ‘total purity of the (S)-pregabalin’ includes both the chemical and enantiomeric purities of (S)-pregabalin.

The term ‘crude (S)-pregabalin’ in the specification refers to pregabalin having HPLC purity of less than about 99%.

If required, pure (S)-pregabalin obtained in step-(c) may be converted into pharmaceutically acceptable salts by conventional methods.

Pharmaceutically acceptable salts of (S)-pregabalin can be prepared in high purity by using the pure (S)-pregabalin obtained by the methods disclosed herein above, by known methods.

The following examples are given for the purpose of illustrating the present disclosure and should not be considered as limitation on the scope or spirit of the disclosure.

### EXAMPLES

### Example 1

(±)-3-(Carbamoylmethyl)-5-methylhexanoic acid (300 gm) was added to a stirred solution of sodium hydroxide (324 gm) in water (1500 ml) at a temperature of −10° C. to −5° C. Bromine (270 gm) was added drop wise to the reaction mass while maintaining the temperature in between −10° C. to −5° C. The mixture was heated at 70-75° C. for 15-20 minutes and then cooled to 20-25° C. To the resulting mass was added oxalic acid to adjust the pH below 1. The mixture was stirred for 20-30 minutes followed by the addition of isobutanol (900 ml) and further stirred for 30 minutes. The layers were separated and the aqueous layer was extracted with isobutanol (600 ml). The total isobutanol layer was combined and pH was adjusted to 5.2±0.2 with triethyl amine. The reaction mass was heated to reflux (75-85° C.) followed by cooling to 0-5° C. and further stirred for 2-3 hours. The resulting solid was filtered, washed with isopropyl alcohol (100 ml) and finally dried at 45-50° C. under vacuum to produce 166 gm of pregabalin (HPLC Purity: 99.2%).

### Example 2

(±)-3-(Carbamoylmethyl)-5-methylhexanoic acid (300 gm) was added to a stirred solution of sodium hydroxide (324 gm) in water (1500 ml) at a temperature of −10° C. to −5° C. Bromine (270 gm) was added to the resulting mass by drop wise addition, while maintaining the temperature at −10° C. to −5° C. The mixture was heated at 70-75° C. for 15-20 minutes and then cooled to 20-25° C. The reaction mass was added to methanesulfonic acid (420 ml) at 20-30° C. and then stirred for 20-30 minutes. This was followed by the addition of isobutanol (900 ml) and stirred for 30 minutes. The resulting layers were separated and the aqueous layer was extracted with isobutanol (600 ml). The isobutanol layer was combined and the pH was adjusted to 5.2±0.2 with triethyl amine. The reaction mass was heated to reflux (75-85° C.) followed by cooling to 0-5° C. and further stirred for 2-3 hours. The separated solid was filtered and washed with isopropyl alcohol (100 ml) and dried at 45-50° C. under vacuum to produce 240 gm of pregabalin (HPLC Purity: 99.1%).

### Example 3

(±)-3-(Carbamoylmethyl)-5-methylhexanoic acid (300 gm) was added to a stirred solution of sodium hydroxide (324 gm) in water (1500 ml) at a temperature in −10° C. to −5° C. Bromine (270 gm) was added to the resulting mass by drop wise addition, while maintaining the temperature at −10° C. to −5° C. The mixture was heated at 70-75° C. for 15-20 minutes and then cooled to 20-25° C. The reaction mass was added to methanesulfonic acid (420 ml) at 20-30° C. The reaction mixture was stirred for 20-30 minutes followed by the addition of isobutanol (900 ml) and stirred for 30 minutes. The resulting layers were separated and the aqueous layer was extracted with isobutanol (600 ml). The isobutanol layer was combined and the pH was adjusted to 6.0±0.2 with triethyl amine. The reaction mass was heated to reflux (75-85° C.), cooled to 0-5° C. and stirred for 2-3 hours. The separated solid was filtered and washed with isopropyl alcohol (100 ml) and dried at 45-50° C. under vacuum to produce 250 gm of pregabalin (HPLC Purity: 99.2%).

### Example 4

### Purification of Pregabalin:

Pregabalin (obtained in example 1, 2 or 3) was dissolved in a mixture of isopropanol and water (1:1) at 75-80° C. and the resulting solution was stirred for 30 minutes. The resulting solution was cooled to 0-5° C. followed by stirring for 4 hours at the same temperature. The solid was filtered, washed with a mixture of chilled isopropanol and then dried under vacuum at 40-45° C. to produce pure pregabalin (HPLC Purity: 99.65%; Yield: 80-85%).

### Example 5

### Purification of (S)-Pregabalin:

Crude (S)-pregabalin (HPLC Purity: 98%) was dissolved in a mixture of methanol and water (0.66:0.34) at 70-75° C. The resulting solution was stirred for 30 minutes at the same temperature and then passed through hyflo to get the solution particle free. The resulting solution was cooled to 0-5° C. followed by stirring for 4 hours at the same temperature. The solid was filtered, washed with a mixture of chilled methanol and water, and then dried under vacuum at 40-45° C. to produce pure (S)-pregabalin (HPLC Purity: 99.9%; Yield: 85-90%).

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A process for the preparation of pregabalin or an (S)-enantiomer thereof, comprising:
a) admixing 3-(carbamoylmethyl)-5-methylhexanoic acid or an (R)-enantiomer thereof with a solution of alkali metal hydroxide in water at a temperature of about −5° C. to about −15° C. to form an admixture;
b) adding bromine to the admixture obtained step-(a) at a temperature of about −5° C. to about −15° C. to form a reaction mixture;
c) heating the reaction mixture obtained in step-(b) at a temperature of about 40° C. to about 100° C. to form a first reaction mass;
d) reacting the reaction mass obtained step-(c) with an organic acid to form an organic acid addition salt of pregabalin;
e) optionally, extracting the organic acid addition salt of pregabalin obtained in step-(d) with an organic solvent to produce an organic layer;
f) treating the organic acid addition salt of pregabalin obtained in step-(d) or the organic layer obtained in step-(e) with a base to form a second reaction mass;
g) heating the second reaction mass obtained in step-(f) at a temperature of about 40° C. to about 100° C. to form a third reaction mass; and
h) isolating substantially pure pregabalin or an (S)-enantiomer thereof from the third reaction mass, and optionally recrystallizing the product obtained from a suitable solvent to produce highly pure pregabalin or an (S)-enantiomer thereof.

2. The process of claim 1, wherein the alkali metal hydroxide used in step-(a) is selected from the group consisting of sodium hydroxide, potassium hydroxide, lithium hydroxide, and cesium hydroxide.

3. The process of claim 1, wherein the admixing in step-(a) is carried out either by adding the 3-(carbamoylmethyl)-5-methylhexanoic acid or its (R)-enantiomer to the alkali metal hydroxide solution, or by adding the alkali metal hydroxide solution to the 3-(carbamoylmethyl)-5-methylhexanoic acid or its (R)-enantiomer; wherein the addition of bromine in step-(b) is carried out at a temperature of about −5° C. to about −12° C. for at least 20 minutes; wherein the reaction mixture in step-(c) is heated at a temperature of about 50° C. to about 90° C. for at least 15 minutes; wherein the first reaction mass obtained after completion of reaction in step-(c) is further cooled at a temperature of below 40° C.; wherein reaction in step-(d) is carried out at a temperature of below 40° C. for at least 15 minutes; wherein the second reaction mass in step-(g) is heated at a temperature of about 50° C. to about 90° C. for at least 15 minutes; wherein the isolation in step-(h) is carried out by cooling, seeding, partial removal of the solvent from the solution, adding an anti-solvent to the solution, or a combination thereof.

4. The process of claim 3, wherein the addition in step-(a) is carried out at a temperature of about −5° C. to about −12° C. for at least 10 minutes; wherein the addition of bromine in step-(b) is carried out at a temperature of about −5° C. to about −10° C. for about 30 minutes to about 4 hours; wherein the reaction mixture in step-(c) is heated at a temperature of about 60° C. to about 85° C. for about 20 minutes to about 2 hours; wherein the first reaction mass obtained after completion of reaction in step-(c) is cooled at a temperature of about 10° C. to about 30° C.; wherein reaction in step-(d) is carried out at a temperature of about 10° C. to about 30° C. from about 20 minutes to about 2 hours; wherein the second reaction mass in step-(g) is heated at a temperature of about 60° C. to about 85° C. from about 20 minutes to about 2 hours; and wherein the isolation in step-(h) is carried out by cooling the solution at a temperature of about 0° C. to about 25° C. for about 20 minutes to about 20 hours.

5. (canceled)

6. The process of claim 1, wherein the alkali metal hydroxide in step-(a) is used in a molar ratio of about 4 to 8 moles per mole of 3-(carbamoylmethyl)-5-methylhexanoic acid or its (R)-enantiomer; and wherein the bromine in step-(b) is used in a molar ratio of about 1 to 3 moles per mole of 3-(carbamoylmethyl)-5-methylhexanoic acid or its (R)-enantiomer.

7. The process of claim 6, wherein the alkali metal hydroxide is used in a molar ratio of about 4.5 to 5.5 moles per mole of 3-(carbamoylmethyl)-5-methylhexanoic acid or its (R)-enantiomer; and wherein the bromine is used in a molar ratio of about 1.05 to 1.25 moles per mole of 3-(carbamoylmethyl)-5-methylhexanoic acid or its (R)-enantiomer.

8.-15. (canceled)

16. The process of claim 1, wherein the organic acid used in step-(d) is selected from the group consisting of oxalic acid, methanesulfonic acid, trifluoroacetic acid, and benzenesulfonic acid; wherein the organic solvent used for extraction in step-(e) is selected from the group consisting of methanol, ethanol, n-propanol, isopropyl alcohol, isobutanol, n-butanol, tert-butanol, amyl alcohol, isoamyl alcohol, hexanol, acetone, methyl ethyl ketone, methyl isobutyl ketone, methyl tert-butyl ketone, ethyl acetate, methyl acetate, isopropyl acetate, tert-butyl methyl acetate, ethyl formate, and mixtures thereof; wherein the base used in step-(f) is an organic or inorganic base; and wherein the recrystallization solvent used in step-(h) is an aqueous alcohol solvent.

17. The process of claim 16, wherein the organic acid is oxalic acid or methanesulfonic acid; wherein the organic solvent used for extraction in step-(e) is selected from the group consisting of isobutanol, n-butanol, tert-butanol, amyl alcohol, isoamyl alcohol, hexanol, and mixtures thereof; wherein the base used in step-(f) is selected from the group consisting of triethyl amine, tributyl amine, ammonia, diisopropyl amine, dimethyl amine and diisopropyl ethyl amine, sodium hydroxide, potassium hydroxide, lithium hydroxide, sodium carbonate, potassium carbonate, and lithium carbonate; and wherein the recrystallization solvent used in step-(h) is an aqueous methanol solvent, wherein the ratio of methanol to water is of 6-8:2-4.

18.-32. (canceled)

33. The process of claim 1, wherein the pregabalin or its (S)-enantiomer obtained in step-(h) is recovered by filtration, filtration under vacuum, decantation, centrifugation, filtration employing a filtration media of a silica gel or celite, or a combination thereof.

34. The process of claim 1, wherein the pregabalin or its (S)-enantiomer obtained has a total purity of about 99% to about 99.99% as measured by HPLC.

35. A process for purifying (S)-pregabalin, comprising:
a) providing a solution of crude (S)-pregabalin in a solvent medium comprising methanol and water, wherein the ratio of methanol to water is of 6-8:2-4;
b) optionally, filtering the solution; and
c) isolating highly pure (S)-pregabalin from the solution.

36. The process of claim 35, wherein the solution in step-(a) is prepared by dissolving crude (S)-pregabalin in the solvent medium; wherein the solution obtained in step-(a) is stirred at a temperature of about 50° C. to about 80° C. for at least 20 minutes; wherein the solution obtained in step-(a) is subjected to carbon treatment or silica gel treatment; and wherein the isolation of pure (S)-pregabalin in step-(c) is initiated by cooling, seeding, partial removal of the solvent from the solution, by adding an anti-solvent to the solution, or a combination thereof.

37. The process of claim 36, wherein the crude (S)-pregabalin is dissolved in the solvent medium at a temperature of about 40° C. to the reflux temperature of the solvent medium; wherein the solution obtained in step-(a) is stirred at a temperature of about 55° C. to about 75° C. from about 30 minutes to about 5 hours; and wherein the isolation in step-(c) is carried out by cooling the solution at a temperature of below 25° C.

38. The process of claim 37, wherein the crude (S)-pregabalin is dissolved in the solvent medium at a temperature of about 50° C. to about 80° C.; and wherein the isolation in step-(c) is carried out by cooling the solution at a temperature of about 0° C. to about 20° C. for about 20 minutes to about 20 hours.

39.-44. (canceled)

45. The process of claim 35, wherein the (S)-pregabalin obtained in step-(c) is recovered by filtration, filtration under vacuum, decantation, centrifugation, filtration employing a filtration media of a silica gel or celite, or a combination thereof.

46. The process of claim 35, wherein the (S)-pregabalin obtained has a total purity of about 99% to about 99.99% as measured by HPLC.
